(19) United States
(12) Patent Application Publication
(10) Pub. No.: US 2022/0250789 A1
(43) Pub. Date: Aug. 11, 2022

(54) PACKAGING BOX

(71) Applicant: Zhejiang Orient Gene Biotech Co., Ltd, Huzhou (CN)

(72) Inventor: Jianqiu FANG, Huzhou (CN)

(21) Appl. No.: 17/582,308

(22) Filed: Jan. 24, 2022

(30) Foreign Application Priority Data
Jan. 25, 2021 (CN) 2021202040850

(51) Int. Cl. B65D 5/52; B65D 5/54; B65D 5/42; G01N 33/569

(52) U.S. Cl. CPC B65D 5/5286; B65D 5/542; G01N 2333/165; G01N 33/56983; B65D 5/4216

(57) ABSTRACT

The present invention provides a packaging box, including a box body; the box body is provided with an insertable zone; a dashed hole is disposed at an edge of the insertable zone. The insertable zone is round and may be completely or partially separated from the box body; there is an insertable zone on the box body. The packaging box provided by the present invention has an insertable zone. The detecting personnel need not find out an extra tubular fixed seat for use. Meanwhile, a packaging box is used to replace a tubular fixed seat, reducing the use cost of the detecting apparatus. Moreover, each lysis tube can be inserted into respective packaging box; and each packaging box is corresponding to respective detecting apparatus. Therefore, it is not easy to cause the misuse of lysis tubes during loading and detection procedure, thereby avoiding wrong detection because the lysis tube is taken by mistake.

### CROSS-REFERENCE TO RELATED APPLICATION

The present invention claims the priority of Chinese patent application No. 201220204085.0, filed on Jan. 25, 2021.

### TECHNICAL FIELD

The present invention relates to the technical field of medical diagnosis, and in particular to a packaging box for a detection reagent.

### BACKGROUND

In the field of rapid detection, to keep the detecting apparatus clean and for the convenience of separating each detecting apparatus, almost all the detecting apparatus are externally provided with a packaging box/packaging bag. In the detection procedure of some detecting apparatus, a lysis tube needs to be inserted into a dedicated tubular fixed seat first, and samples are added to the lysis tube, then the lysis tube is taken down and liquid therein is added on a detecting apparatus for detection. There are several problems in the procedure: 1. such a tubular fixed seat used for fixing the lysis tube is exclusive of the packaging box/packaging bag; therefore, the detecting personnel need to separately find out a piece of fixed seat for use, which is not very convenient; 2. if the packaging box/packaging bag contains an independent tubular fixed seat, the use cost, package and transportation of the detecting apparatus are increased; 3. the tubular fixed seat is provided with a plurality of holes for fixing the tube; generally, a piece of tubular fixed seat is inserted with a plurality of lysis tubes, and each lysis tube contains different samples; since these holes on the tubular fixed seat are close with each other, the detecting personnel are prone to taking the lysis tube by mistake during detection, leading to a wrong detection result.

Therefore, it needs to provide an easier, more convenient and more cheaper packaging box of a detection reagent. Such kind of packaging box can further exert an effect of auxiliary detection besides packaging the detection reagent.

### SUMMARY

The technical problem to be solved by the present invention is to provide a packaging box of a detection reagent, thus solving the above technical problem.

The present invention provides a packaging box, including a box body, where, the box body is provided with an insertable zone. In some embodiments, the insertable zone is used for inserting a test tube or a tube body provided with a sample treatment fluid.

In some embodiments, a dashed hole or a scribed line is disposed at an edge of the insertable zone. In this way, the dashed hole or scribed line is used for opening during insertion. Such a mode may be configured in many ways. It is available that the inserted zone is readily torn off to form a hole when the insertable zone is inserted by a test tube or a tube body.

In some embodiments, the insertable zone is round, or in any other shape, such as, square, diamond, oval.

In some embodiments, the insertable zone may be completely or partially separated from the box body, or on the box or is part of the box.

In some embodiments, there is an insertable zone on the box body. In some embodiments, there are two or above insertable zones on or in the box body. In some embodiments, a space between the two insertable zones is less than 5 cm. In some embodiments, the insertable zone is located close to an edge position in the box body.

In some embodiments, a storing space is disposed inside the box body; and the storing space may be used for holding a detecting apparatus or devices, and/or a lysis tube, and/or a dropper, and/or a cotton swab. The detecting apparatus here is a latter flow detecting apparatus. The lysis tube includes reagents capable of lysing viruses, and these reagents exist in a state of solution, for example, a state of aqueous solution.

In some embodiments, the lysis reagent includes titanium dioxide, a buffer solution, a surfactant and a preservative. In some embodiments, the titanium dioxide is nano-TiO2. In some embodiments, the nano-TiO2 has a content of 0.01%-0.1%. In some embodiments, the buffer solution includes sodium chloride, phosphate and casein sodium. In some embodiments, the lysis reagent includes 0.15-0.45 mol/L sodium chloride, 1.15-2.9 g/L phosphate and 0.5% casein sodium. In some embodiments, the surfactant is Triton X-100. In some embodiments, the preservative is Proclin 300. In some embodiments, the lysis solution includes borax, casein sodium, Triton, NaCl and Proclin. In some embodiments, the lysis solution also includes borax, casein sodium, Triton, NaCl, Proclin and N6-(2-hydroxyethyl)adenosine.

Further, instructions of the detecting apparatus and the packaging box are printed on the back of the box body.

The present invention has the following beneficial effects: the packaging box provided by the present invention has an insertable zone. The detecting personnel need not find out an extra tubular fixed seat for use. Meanwhile, a packaging box is used to replace a tubular fixed seat, reducing the use cost of the detecting apparatus. Moreover, each lysis tube can be inserted into respective packaging box; and each packaging box is corresponding to respective detecting apparatus. Therefore, it is not easy to cause the misuse of lysis tubes during loading and detection procedures, thereby avoiding wrong detection because the lysis tube is taken by mistake.

### BRIEF DESCRIPTION OF THE DRAWINGS

FIG. 1 is a structure diagram showing a packaging box.

FIG. 2 is a local enlarged diagram showing an “A” zone in FIG. 1.

FIG. 3 is a structure diagram showing another packaging box.

FIG. 4A is a structure of a lysis tube, FIG. 4B is the tope view of the sealing film for sealing the lysis tube; FIG. 4C is a structure of a doper for inserting in the tube for dropping the solution into the sample pad or sample hole 902, FIG. 4B is a structure of a detecting devices with result window 901 and a sample hole for receiving the sample 902 and a hold part for being handed.

FIG. 5A is embodiment of a inserting zone with tear-off line, FIG. 5B is another embodiment of a inserting zone with a cross tear-off line as to define the inserting zone; FIG. 5C is also another embodiment of a inserting zone with a cross tear-off line.

FIG. 6A is a sample of the present invention with two inserting zones 50, 51 near the end of the package box; FIG. 6B is one of a embodiments of a tube is inserted into the zone 51; FIG. 6C is one of a embodiments for a method for how to use the box for home test that is printed on the box.

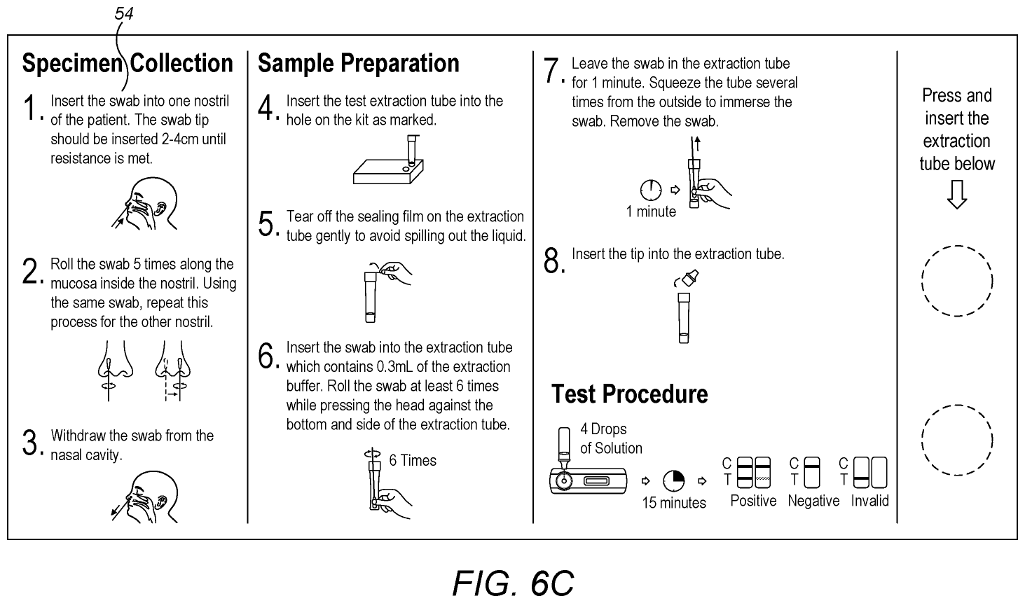

### DETAILED DESCRIPTION OF THE EMBODIMENTS

The structures or technical terms used in the present invention are further described in the following. Unless otherwise indicated, they are understood or interpreted according to ordinary terms and definitions in the art.

Detection

Detection denotes assaying or testing whether a substance or material exists. The substance or material, for example, is but not limited to, chemicals, organic compounds, inorganic compounds, metabolites, drugs or drug metabolites, organic tissues or metabolites of organic tissues, nucleic acid, proteins or polymers. Moreover, detection denotes testing the number of a substance or material. Further, assay also denotes immunoassay, chemical detection, enzyme detection and the like.

Samples

In this present invention, the samples collected by the detection apparatus include biological liquid. Initial state of the sample may be liquid, solid or semi-solid; solid or semi-solid samples can be converted to liquid samples using any appropriate method, such as mixing, crushing, macerating, incubating, dissolving or digesting; and then the liquid samples are poured into a collection chamber; a testing element is used to test whether an analyte is contained in the samples. Samples can be derived from human bodies, animals, plants, the nature, and the like. Samples derived from human bodies, for example, may be blood, serum, urine, cerebrospinal fluid, sweat, lymphatic fluid, saliva, gastric juice and other liquid samples; faeces, hair, cutin, dental calculus, finger nails/toe nails and other solid and semi-solid samples. Samples derived from plants, for example, may be roots, stems, leaves and other solid samples; tissue fluids and cell fluids prepared by roots, stems, leaves, and other liquid or semi-solid samples. Samples derived from the nature, for example, may be rainwater, river water, seawater, underground water, and other liquid samples; soil, rock, mineral, petroleum and other solid or semi-solid samples.

Samples described here may include throat swab samples, nose swab samples, or sputum, lung fluid, and the like.

Testing Element

The “testing element” used herein refers to an element that can be used to detect an analyte in a sample to be detected. Such a testing element can be based on any technical principles, such as immunology, chemistry, electricity, optics, molecular science, nucleic acids, physics, etc. The testing element used herein can be one or a combination of more than two testing elements. The testing element has a detection zone used for showing a detection result; after detection, the detection zone shows a detection result.

A common type of the testing element is a test paper or a latter flow strip. The detection of the test paper for samples to be detected can be based on immunoassay or chemical analysis. The analysis mode of non-competition law or competition law can be adopted for test papers. A test paper successively contains a sample application area, a reagent area and a testing area.

Samples are added to the sample application area and flow to the reagent area through capillary action; and are reacted with a reagent in the reagent area. Then, samples continue to flow to the testing area through capillary action to observe whether the testing area generates a signal or no signal, thus indicating the presence or the analyte in the sample to be detected or not. For example, if a Test Line (T line) appears in the testing area, it indicates absence of the analyte in the sample to be detected; if there is no T line in the testing area, it indicates presence of the analyte in the sample to be detected. Some test paper is further provided with a control area, and the control area is located behind the testing area. Samples continue to flow to the control area after flowing through the testing area; and the control area is used to judge whether the test result of the testing area is valid. For example, in some test paper, the test result in the testing area is indicated valid only when a Control Line (C line) appears in the control area. Otherwise, the test result in the testing area is indicated invalid. In this present invention, when the test result of the testing element can be read only through the testing area, the detection zone of the testing element is the above testing area; when the test result of the testing element needs to be judged in combination with the signals generated by the testing area and the control area, the detection zone of the testing element includes a testing area and a control area. Of course, in some cases, when the detection results of the testing element need to be judged in combination with signals from other areas, the detection zone also includes the corresponding areas. That is, a complete detection result can be read through the detection zone of the testing element in this present invention. Generally, the testing element at least includes a testing area to obtain whether there exists an analyte in a sample. For example, by appearance or change of color, generally, color change can be visible by naked eyes, and also can be read by scanning equipment, or a test result is read by shooting, or a fluorescence, or a ray occurs, or the like. The test result in the detection zone is read by instruments and equipment.

The testing element generally consists of porous water absorbing materials, for example, filter paper, glass fibers, polyester films, nylon films, paper and non-woven fabrics or any other water absorbing material. Generally, the testing area consists of water absorbing materials, for example, water absorbing films, for example, nitrocellulose membranes, nylon films, and the like.

In some embodiments, the test line on the test stripe includes an immobilized antibody; and a labeled pad includes another antibody; and the antibody can specifically bind to an analyte in a sample. For example, when a coronavirus is detected, the antibody binding to antibodies is treated on the labeled pad, and the antibody is coupled with a colored labeled substance, for example, gold-labeled or latex particles; the detection line is immobilized with an antibody to specifically bind an antigen or the antibody on the labeled pad.

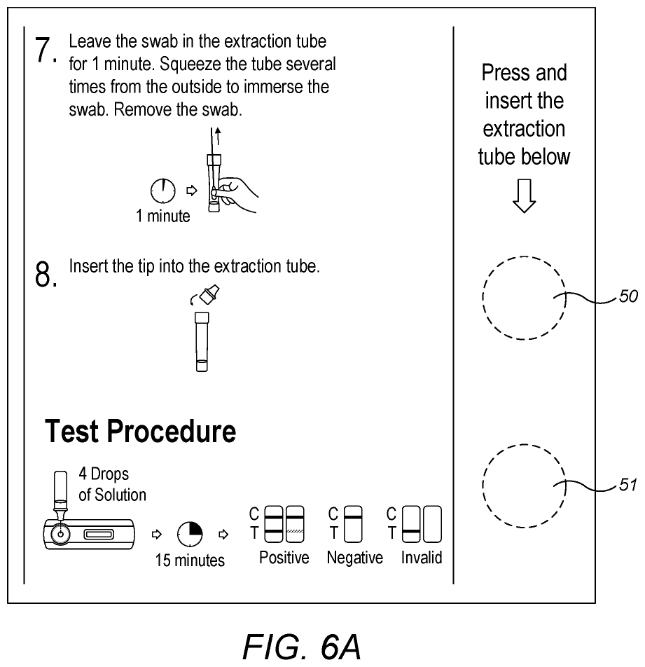

In some embodiments, such kind of test stripe may be configured on the apparatus as shown in FIG. 4C; the apparatus is composited by two sandwich plates; and the plate is provided with a window 901 which is used to observe whether a detection line appears on the detection zone of the test stripe. A loading well 902 is disposed upstream of the detection zone, and used for sample delivery; and the sample is delivered from the lysis tube. In some embodiments, a portion for holding is specifically disposed on the apparatus to avoid the splashing of samples during loading process. Such portion is particularly helpful to the products for domestic detection. During the home detection for a similar disease, when a sample is dropwisely added to a loading well, the sample needs to be put on a plane, generally a table, a chair or a flat surface at home; and the apparatus needs to be taken out from the packaging box, the portion 903 for holding is wider than the window and the area for adding the same 902 and thus, is held easily. In addition, the portion for holding is slightly higher than the window and the area for adding the same 902. In this way, when the sample is put on the plane for detection, the window 901 and the area for adding the same 902 contact with the table, but there is a certain distance between the portion for holding and the table, for example, a height of a finger, e.g., 2 cm and 1.5 cm. With such a configuration, the apparatus may be taken with fingers conveniently and discarded.

Analyte

For example, the analyte detected by the present invention includes but not limited to creatinine, bilirubin, nitrite, proteins (nonspecific), hormones (for example, human chorionic gonadotropin, progesterone, follicle-stimulating hormone, etc.), blood, leucocyte, sugar, heavy metals or toxins, bacterial substances (such as, proteins or carbohydrates against specific bacteria, for example, Escherichia coli. 0157:H7, Staphylococcus, Salmonella, Fusiformis genus, Camyplobacter genus, L. monocytogenes, Vibrio, or Bacillus cereus) and substances associated with physiological features in a urine sample, such as, pH and specific gravity. The chemical analysis of any other clinical urine may be conducted by means of a lateral cross-flow detection way and in combination with the apparatus of the present invention.

Of course, the analyte may further include some infectious microorganisms, such as, bacteria and viruses. For example, viruses may be contagious viruses, such as, hepatitis virus, AIDS virus, influenza virus and coronavirus. In case of detection, both nucleic acid components of these viruses and antigens may be detected as well as collected, or antibodies in samples are detected.

Examples capable of utilizing the analyte of the present invention include some hapten substances, these haptens include drugs (e.g., drug of abuse). “Drug of Abuse” (DOA) refers to using a drug (playing a role of paralyzing the nerves usually) not directed to a medical purpose. Abuse of these drugs will lead to physical and mental damage, produce dependency, addiction and/or death. Examples of DOA include cocaine, amphetamine AMP (for example, Black Beauty, white amphetamine table, dextroamphetamine, dextroamphetamine tablet, and Beans); methylamphetamine MET (crank, methamphetamine, crystal, speed); barbiturate BAR (e.g., Valium□, Roche Pharmaceuticals, Nutley, and New Jersey); sedative (namely, sleep adjuvants); lysergic acid diethylamide (LSD); depressor (downers, goofballs, barbs, blue devils, yellow jackets, methaqualone), tricyclic antidepressants (TCA, namely, imipramine, Amitryptyline and Doxepin); methylene dioxymetham-phetamine (MDMA); phencyclidine (PCP); tetrahydrocannabinol (THC, pot, dope, hash, weed, and the like). Opiates (namely, morphine MOP or, opium, cocaine COC; heroin, oxycodone hydrochloride); antianxietics and sedative hypnotics, antianxietics are drugs for alleviating anxiety, tension, fear, stabilizing emotion and having hypnosis and sedation, including benzodiazepines (BZO), non-typical BZs, fusion dinitrogen NB23Cs, benzoazepines, ligands of a BZ receptor, open-loop BZs, diphenylmethane derivatives, piperazine carboxylates, piperidine carboxylates, quinazoline ketones, thiazine and thiazole derivatives, other heterocyclic, imidazole sedatives/analgesics (e.g., oxycodone hydrochloride OXY, metadon MTD), propylene glycol derivatives, mephenesin carbamates, aliphatic compounds, anthracene derivatives, and the like. The detection device of the present invention may be used for detecting drugs which belong to medical use but is easy to be taken excessively, such as tricyclic antidepressants (Imipramine or analogues), acetaminophen and the like. These drugs are absorbed by a human body and then decomposed into different small molecules; and these small molecules exist in blood, urine, saliva, sweat and other body fluid or the above small molecules exist in a portion of body fluid.

Packaging Box and Insertion Hole

The technical solution adopted herein is a packaging box, including a box body 10; a storing space is disposed inside the box body 10; and the storing space may be used for holding one or more of detecting apparatus, testing element, lysis tube, dropper, and cotton swab. For example, when a testing element is used to detect a coronavirus antigen, the packaging box includes a testing element, and an antibody is treated on the testing element, and the antibody may specifically bind a novel coronavirus fragment (antigen); the lysis tube contains an agent capable of lysing the novel coronavirus; for example, in some embodiments, the lysis reagent includes titanium dioxide, a buffer solution, a surfactant and a preservative. In some embodiments, the titanium dioxide is nano-TiO2. In some embodiments, the nano-TiO2 has a content of 0.01%-0.1%. In some embodiments, the buffer solution includes sodium chloride, phosphate and casein sodium. In some embodiments, the buffer solution includes 0.15-0.45 mol/L sodium chloride, 1.15-2.9 g/L phosphate and 0.5% casein sodium. In some embodiments, the surfactant is Triton X-100. In some embodiments, the preservative is Proclin 300. In some embodiments, the lysis solution includes borax, casein sodium, Triton, NaCl and Proclin. In some embodiments, the lysis solution also includes borax, casein sodium, Triton, NaCl, Proclin and N6-(2-hydroxyethyl)adenosine. In some embodiments, a sampling device is further included, for example, sampling is performed with a flocking cotton swab.

In some embodiments, the box body is further provided with an insertion area for inserting the lysis tube. The insertion area may be disposed in anyplace of the box body, a margin area, a central area, front or back of the box body is available. Generally, an operating manual is on the back of the box body, and the insertion area is disposed near the operating manual. The insertable zone may enable the lysis tube to be inserted into the upright area. Such an insertable zone may be disposed in many ways, for example, the box body 10 has a round hole 20 on the edge, and the hole 20 may directly enable the lysis tube to be inserted into the hole and keep upright. In case of detection, in particular to domestic detection, the lysis test tube may be taken out of the packaging box and inserted into the hole and kept upright. The lysis tube taken out of the packaging box is sealed at this time and the lysis solution is sealed in the lysis tube. The sealed test tube may be opened and not sealed before insertion, for example, the lysis tube 70 shown in FIG. 4A; the lysis tube contains a lysis reagent or lysis solution; the lysis tube is sealed by a sealing film 701, for example, sealed by aluminum foil. The sealing film 701 may be torn off, and then inserted into the hole of the box body and kept upright. Then, the sampling device is taken out of the packaging box, for example, a cotton swab, a flocking cotton swab is used to absorb a sample, for example, saliva, nasal mucus or throat sputum, from an oral cavity, a throat and a nasal cavity; after sampling, the sampling head of the cotton swab is inserted into the lysis tube, such that the sample on the sampling head is lysed and dissolved into the lysis solution; the lysis solution may lyse virus, bacteria or others in the sample, thus forming an antigen fragment.

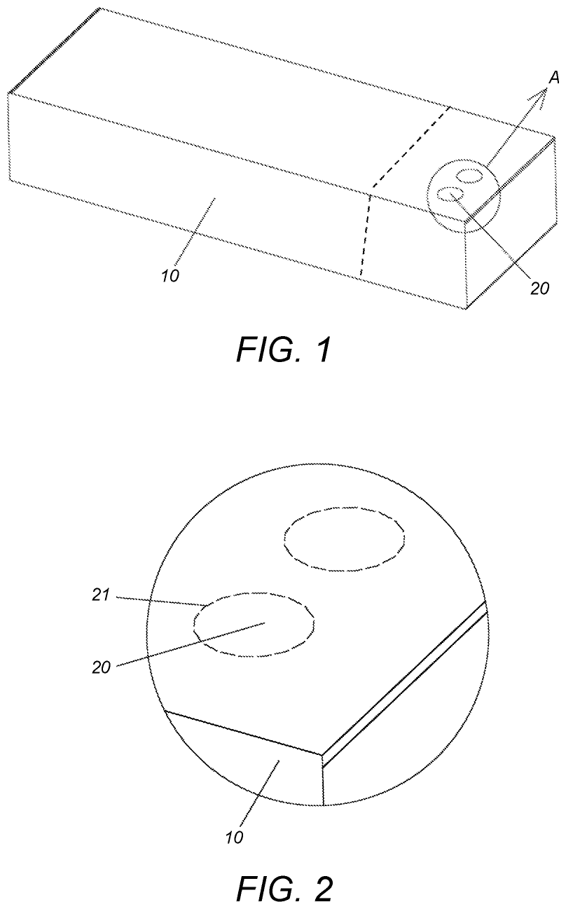

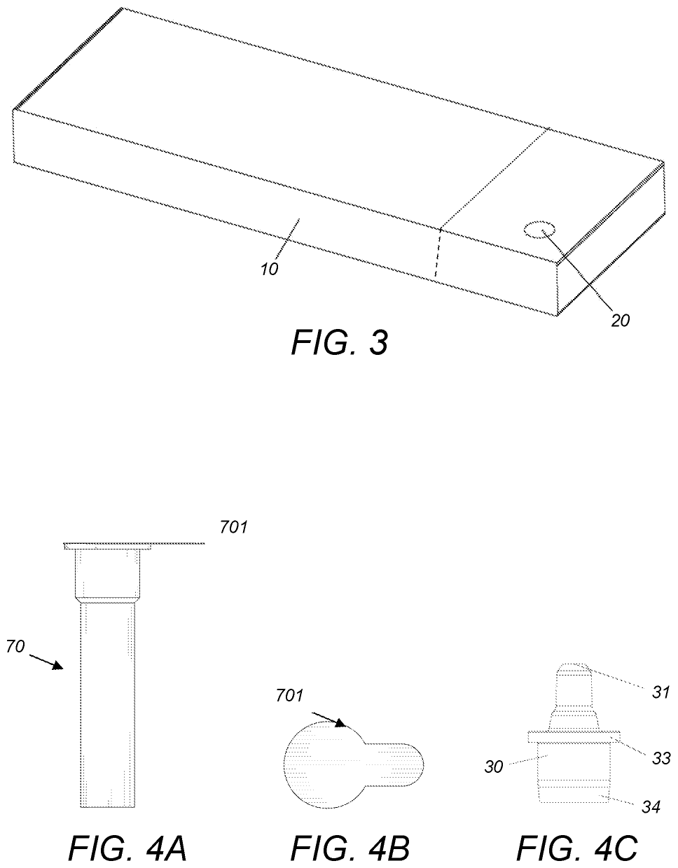

In some embodiments, the area of the hole where the lysis tube is inserted is separated from the area loaded with the reagent, lysis tube and sampling device, for example, the area divided by the dotted line in FIG. 1. In this way, the area where the lysis tube is inserted and the area packaged with the reagent are two portions in the box body, are not in contact with each other and in an isolated state. In some embodiments, the hole in the insertion area is sealed first, but has a tear-off line for division; the tear-off line 21 is to form an area for inserting a tube body in the box body. For example, as shown in FIG. 2, when the lysis tube is not inserted, the area 20 of the box body is blocked; when the lysis tube needs to be inserted, a pressure is exerted directly from the bottom of the lysis tube to the area 20, the area 20 on the box body is sunken or separated due to the existence of the tear-off line, such that the lysis tube is inserted into the hole with the pressure, thus keeping upright.

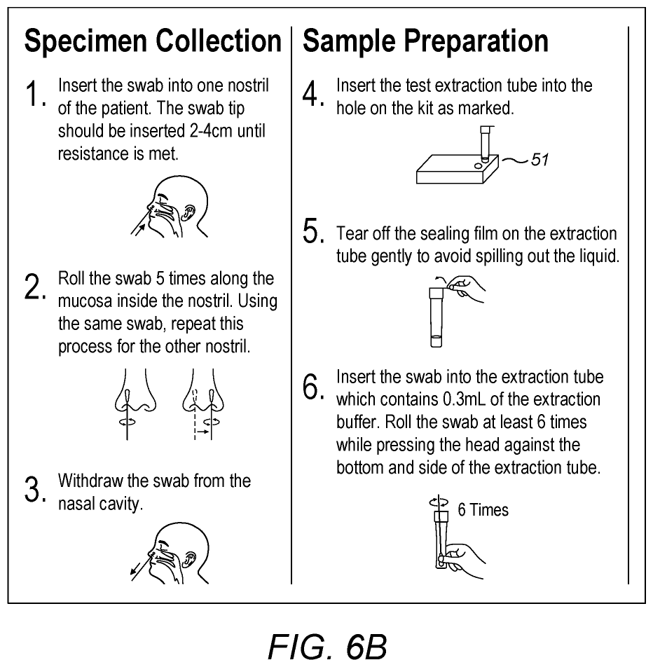

In some embodiments, the inserted area may be separated by a plurality of ways, for example, as shown in FIG. 5B or FIG. 5A, a plurality of tear-off lines are disposed at the insertion area, such that the insertion area will be separated or torn off more easily, and the tube body is kept upright on the packaging box during insertion process. Of course, the example of FIG. 5C may be available; the tear-off line is two crossed lines. Such a configuration mode may also achieve that the inserted area is sunken or the tear-off line is broken, such that the tube body is kept upright on box body during insertion process.

Such an insertion hole is directly disposed on the packaging box, which is particularly convenient in operation. Especially, in case of domestic detection or outdoor detection, the lysis tube needs to be kept upright, such that the sampling device is inserted into the lysis tube. The packaging box in this present invention serves as a base, and the base is provided with an insertion hole, such that the lysis tube may be directly inserted into the hole and kept upright. For example, in case of self-detection in the outdoor, a sampling device, for example, a cotton swab is provided generally as well as the detecting apparatus shown in FIG. 4C, the lysis tube shown in FIG. 4A, and the dropper shown in FIG. 4C, there are so many accessories. Therefore, it is difficult for the unfamiliar personnel to how to use. If the lysis tube may be taken and directly inserted into the hole 20, the operation is simple and convenient, avoiding complex operation.

For example, the box body 10 may be provided with an insertable zone 20; lysis tube, dropper and such kind of device may be directly inserted into the insertable zone 20 of the box body 10 after being taken, thus achieving fixation. After the lysis tube is inserted into the insertable zone 20, liquid in the lysis tube is hardly spilt; and the fixation of the lysis tube facilitates that the sample to be detected is added to the lysis tube by the detecting personnel. When more than one person require detection together, the lysis tubes are inserted into the holes of respective packaging boxes; after detection, the respective detection results are viewed to avoid the confusing of each lysis tube.

Moreover, by using the packaging box of the present invention, the detecting personnel need not find out an extra tubular fixed seat for use. Meanwhile, a packaging box is used to replace a tubular fixed seat, reducing the use cost of the detecting apparatus. Moreover, each lysis tube can be inserted into respective packaging box; and each packaging box is corresponding to respective detecting apparatus. Therefore, it is not easy to cause the misuse of lysis tubes during loading and detection procedure, thereby avoiding wrong detection because the lysis tube is taken by mistake.

Preferably, a dashed hole 21 is disposed at an edge of the insertable zone 20; the dashed hole 21 enables the insertable zone 20 on the box body 10 to be inserted more easily, which is convenient for the detecting personnel. Meanwhile, after the lysis tube is inserted into the insertable zone 20, the insertable zone 20 will be completely or partially separated from the box body 10 along with the dashed hole 21. The “dashed” may be configured by an easily tear-off way, for example, a device is used to pierce the position of the dashed line, thus forming a place which is torn off easily.

Generally, the insertion end of such a device, a lysis tube is round. Therefore, preferably, the insertable zone 20 is round. After the lysis tube is inserted such that the insertable zone 20 is separated, a round hole will be reserved on the box body 10. The insertion end of the lysis tube is matched with the round hole, ensuring the stability of the lysis tube after insertion.

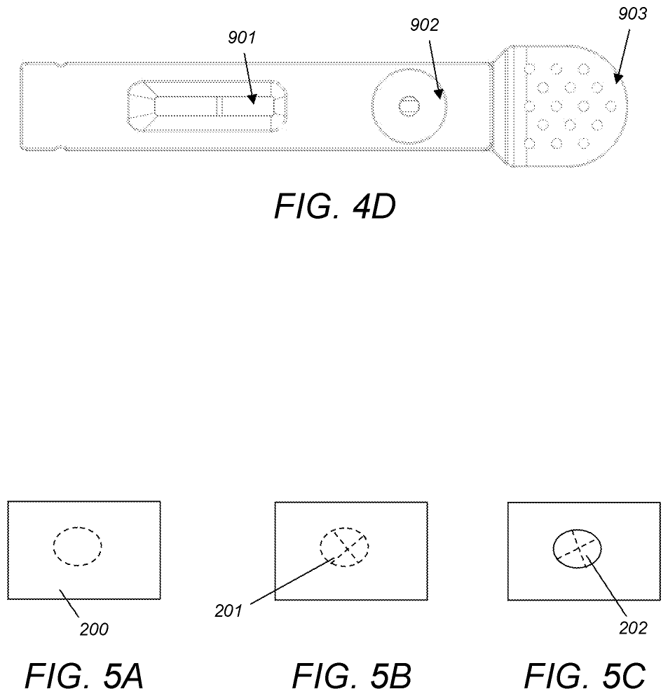

Preferably, by reference to FIG. 3, there is one insertable zone 20 on the box body 10. In general, such a packaging box has a relatively small volume and only holds a set of detecting apparatus. Therefore, an insertable zone 20 is only preserved on the box body, for example, the packaging box includes a detecting apparatus for the detection of saliva.

Preferably, by reference to FIG. 1, there are two insertable zones 20 on the box body 10. In general, such a packaging box has a relatively large volume and may hold two sets of detecting apparatus. Therefore, two insertable zones 20 are preserved on the box body, for example, the packaging box includes a detecting apparatus for the detection of intranarial substances, and two nostrils need to be detected separately. Further, a space between the two insertable zones 20 is less than 5 cm.

In some other embodiments, there are three or above insertable zones 20 on the box body 10. The manufacturer may design the number according to the use requirements of the detecting personnel. Preferably, the insertable zone 20 is located close to an edge position in the box body 10. Preferably, instructions of the detecting apparatus and the packaging box are printed on the back of the box body 10, used for informing the detecting personnel of how to use it.

In some other embodiments, the packaging box is made of a paper material, which is convenient for printing and making the insertion hole.

In some embodiments, the packaging box is shown in FIGS. 5A-5C, the operating process is printed on the background of the packaging box, of which, the step 4 is the operation diagram 51, namely, the lysis tube is inserted into the hole of the packaging box, as shown in FIG. 5B. The sealing film is not torn off this time. Step 5 is to tear off the sealing film, step 6 is to insert the cotton swab for sample lysis; step 7 is to wait for a minute for full lysis; step 8 is to cover the dropper, as shown in FIG. 4C. The dropper has an opening where a main body 30 is inserted into the lysis tube, an outlet 31, and a ring 33, which is aimed at limiting the insertion depth when the cotton swab is inserted into the opening of the lysis tube. After the dropper is inserted, the tube body of the lysis tube is soft and thus may be squeezed, such that the lysis solution is dropwisely added on the loading well 902 to complete the detection. The packaging box has two insertion holes 50,51, as shown in FIG. 5A. Such two holes are kept upright such that the lysis tube is inserted.

The present invention as shown and set forth in this text may be achieved in case of lacking any element and limitation disclosed herein specifically. Terms and expression methods used herein are used for description, but not for limitation. Further, it is undesired that any equivalent of the features or a portion thereof as shown or set forth herein is excluded in the use of these terms and expression methods; moreover, a person skilled in the art should realize that various modifications are feasible within the scope of the present invention. It should therefore be understood that, although the present invention has been specifically disclosed through various embodiments and optional characteristics, modifications and variations of concepts described herein may be employed by ordinary technicians skilled in the art, and these modifications and variations are considered to fall within the scope of the present invention defined by the attached claims.

The content of articles, patents, patent applications and all other documents and electronically available information described or documented herein is incorporated in the full text to some extent for reference, as if each individual publication is specifically and individually pointed out for reference. The Applicant reserves the right to incorporate any and all materials and information from this article, patent, patent application or other documents into the present application.

1. A packaging box, comprising a box body, wherein, the box body is provided with an insertable zone.

2. The packaging box according to claim 1, wherein, a dashed hole is disposed at an edge of the insertable zone.

3. The packaging box according to claim 1, wherein, the insertable zone is round, oval or square.

4. The packaging box according to claim 1, wherein, the insertable zone may be separated from the box body.

5. The packaging box according to claim 1, wherein, there is an insertable zone on the box body.

6. The packaging box according to claim 1, wherein, there are two or above insertable zones on the box body.

7. The packaging box according to claim 6, wherein, a space between the two insertable zones is less than 5 cm.

8. The packaging box according to claim 1, wherein, the insertable zone is located close to an edge position in the box body, or in a middle position of a shell.

9. The packaging box according to claim 1, wherein, a storing space is disposed inside the box body; and the storing space may be used for holding a detecting apparatus, and/or a lysis tube, and/or a dropper, and/or a sampling device.

10. The packaging box according to claim 9, wherein, instructions of the detecting apparatus and the packaging box are printed on the back of the box body.

11. The packaging box according to claim 10, wherein, the insertable zone is located on the back of the box body.

12. The packaging box according to claim 9, wherein, the detecting apparatus comprises a latter flow test stripe.

13. The packaging box according to claim 9, wherein, the lysis tube comprises a reagent for lysing virus.

14. The packaging box according to claim 9, wherein, the virus is a novel coronavirus.

15. The packaging box according to claim 1, wherein, the edge of the insertable zone is provided with a tear-off line which is readily torn off.

16. The packaging box according to claim 1, wherein, the insertable zone is a hole or a plurality of holes.

17. The packaging box according to claim 1, wherein, the packaging box is made of a paper material.

18. The packaging box according to claim 9, wherein, the insertable zone is configured to be insert the lysis tube, such that the lysis tube is kept upright on the packaging box.

19. The packaging box according to claim 13, wherein, the reagent for lysing virus comprises titanium dioxide, a buffer solution, a surfactant and a preservative.

20. The packaging box according to claim 13, wherein, the reagent for lysing virus comprises borax, casein sodium, Triton, NaCl and Proclin.
